(19) United States
(12) Patent Application Publication
(10) Pub. No.: US 2018/0099082 A1
(43) Pub. Date: Apr. 12, 2018

(54) Intravenous Flushing System

(71) Applicants: Sandra Macasieb, Las Vegas, NV (US); Armand Macasieb, Las Vegas, NV (US)

(72) Inventors: Sandra Macasieb, Las Vegas, NV (US); Armand Macasieb, Las Vegas, NV (US)

(21) Appl. No.: 15/287,847

(22) Filed: Oct. 7, 2016

(51) Int. Cl. A61M 5/14; A61M 5/00; A61M 39/16

(52) U.S. Cl. CPC A61M 5/14; A61M 2005/1403; A61M 39/162; A61M 5/001

(57) ABSTRACT

An intravenous flushing system includes an intravenous tube that has an input port. A cleaning unit is provided and the cleaning unit contains a saline solution. The cleaning unit is selectively coupled to the input port. The cleaning unit urges the saline solution through the intravenous tube thereby facilitating the intravenous tube to be cleaned. A sterilizing unit is removably coupled to the cleaning unit. The sterilizing unit is infused with an antiseptic. The sterilizing unit engages the input port. Thus, the antiseptic sterilizes the input port.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed under 37 CFR 1.97 and 1.98

The disclosure and prior art relates to flushing devices and more particularly pertains to a new flushing device for sterilizing and flushing an intravenous tube.

### BRIEF SUMMARY OF THE INVENTION

An embodiment of the disclosure meets the needs presented above by generally comprising an intravenous tube that has an input port. A cleaning unit is provided and the cleaning unit contains a saline solution. The cleaning unit is selectively coupled to the input port. The cleaning unit urges the saline solution through the intravenous tube thereby facilitating the intravenous tube to be cleaned. A sterilizing unit is removably coupled to the cleaning unit. The sterilizing unit is infused with an antiseptic. The sterilizing unit engages the input port. Thus, the antiseptic sterilizes the input port.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an intravenous flushing system according to an embodiment of the disclosure.

FIG. 2 is an exploded perspective view of an embodiment of the disclosure.

FIG. 3 is a right side view of an embodiment of the disclosure.

FIG. 4 is a cross sectional view taken along line 4-4 of FIG. 3 of an embodiment of the disclosure.

FIG. 5 is a perspective in-use view of an embodiment of the disclosure showing a sterilizing unit being uncoupled from an intravenous tube.

FIG. 6 is a perspective in-use view of an embodiment of the disclosure showing a sterilizing unit being coupled to an intravenous tube.

FIG. 7 is a perspective in-use view of an embodiment of the disclosure showing a cleaning unit being uncoupled from an intravenous tube.

FIG. 8 is a perspective in-use view of an embodiment of the disclosure showing a cleaning unit being coupled to an intravenous tube.

### DETAILED DESCRIPTION OF THE INVENTION

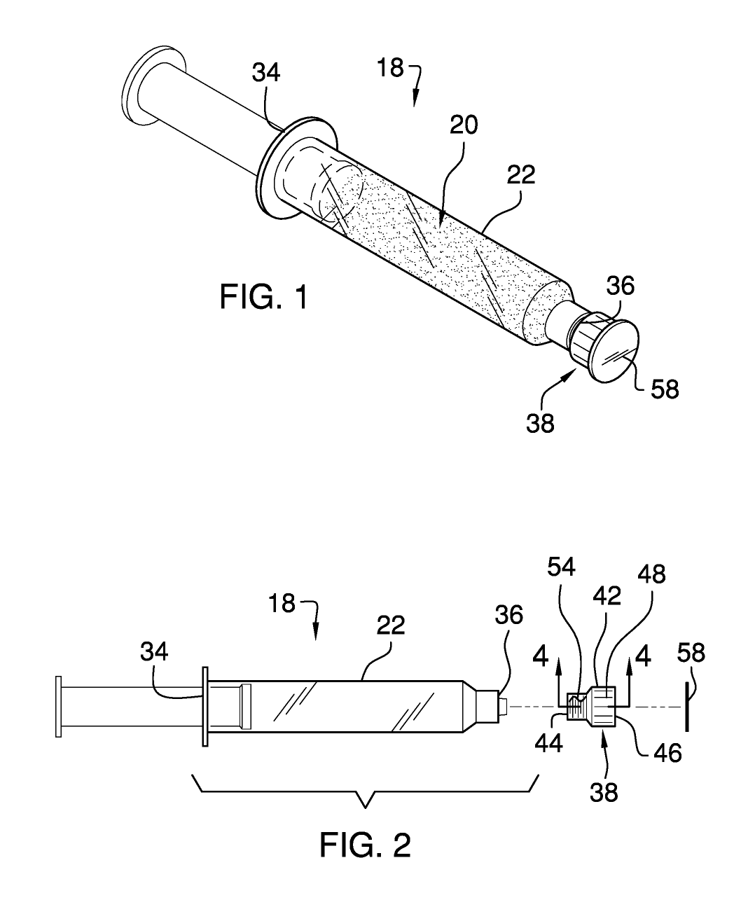

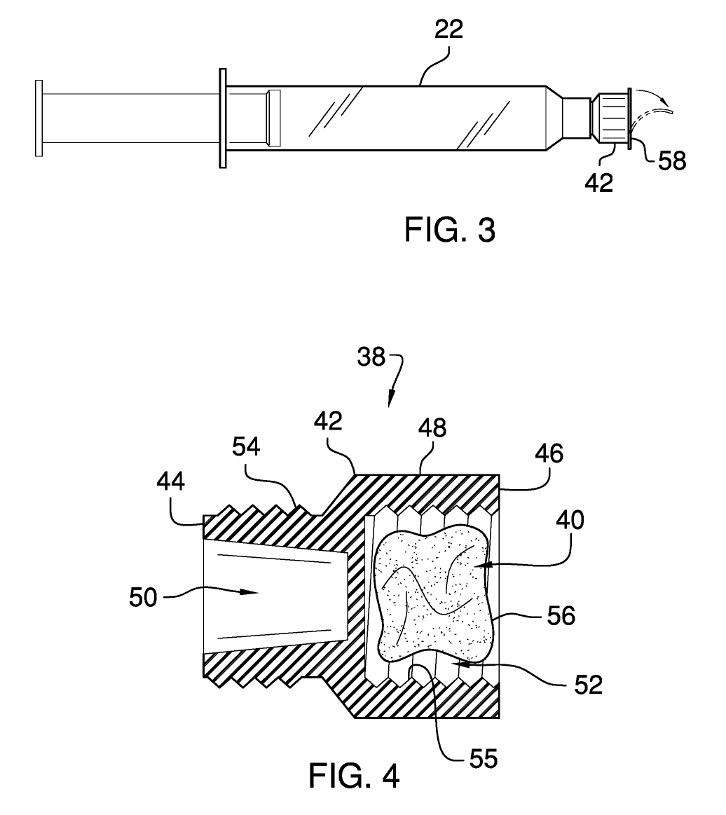

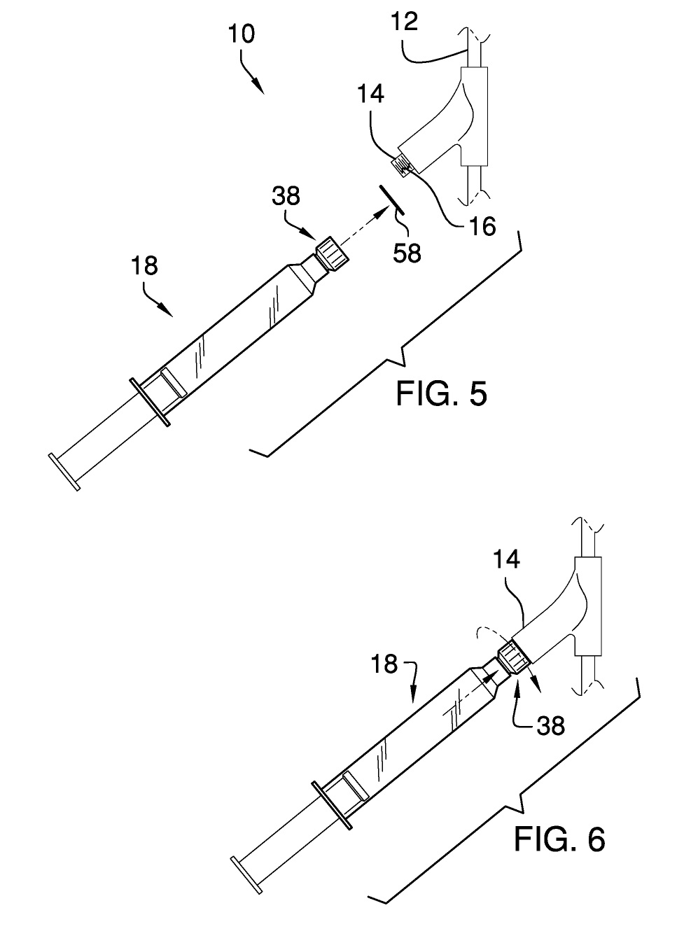

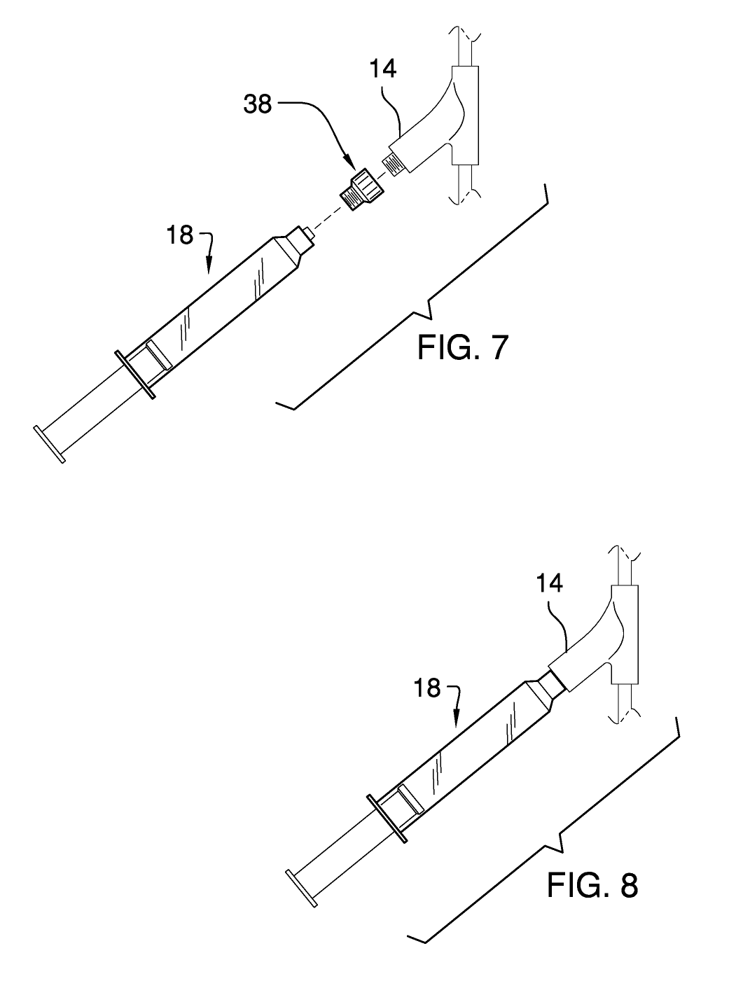

With reference now to the drawings, and in particular to FIGS. 1 through 8 thereof, a new flushing device embodying the principles and concepts of an embodiment of the disclosure and generally designated by the reference numeral 10 will be described.

As best illustrated in FIGS. 1 through 8, the intravenous flushing system 10 generally comprises an intravenous tube 12 that has an input port 14. The input port 14 has an outer surface 16. The outer surface 16 is threaded. The intravenous tube 12 is employed in a sterile, medical environment.

A cleaning unit 18 is provided and the cleaning unit 18 contains a saline solution 20. The cleaning unit 18 is selectively coupled to the input port 14. The cleaning unit 18 urges the saline solution 20 through the intravenous tube 12. Thus, the intravenous tube 12 is flushed thereby removing debris and other contaminants from the intravenous tube 12. The cleaning unit 18 is contained in a sterile bag. The cleaning unit 18 is selectively removed from the sterile bag thereby facilitating the cleaning unit 18 to be employed on the sterile, medical environment.

The cleaning unit 18 comprises a syringe 22 that has a first end 24, a second end 26 and an outer wall 28 extending therebetween. Each of the first end 24 and the second end 26 is open. The syringe 22 contains the saline solution 20. A nipple 30 extends away from the second end 26 and the nipple 30 is in fluid communication with the syringe 22. The nipple 30 selectively engages the input port 14. Thus, the syringe 22 is in fluid communication with the intravenous tube 12.

A plunger 32 is provided that has a primary end 34 and a secondary end 36. The plunger 32 is slidably positioned in the syringe 22 and the plunger 32 may be manipulated. The secondary end 36 frictionally engages the outer wall 28 of the syringe 22. Moreover, the secondary end 36 forms a fluid impermeable seal with the outer wall 28. The secondary end 36 is urged toward the second end 26 of the syringe 22 thereby urging the saline solution 20 into the intravenous line.

A sterilizing unit 38 is provided and the sterilizing unit 38 is removably coupled to the cleaning unit 18. The sterilizing unit 38 is infused with an antiseptic 40. The sterilizing unit 38 engages the input port 14. Thus, the antiseptic 40 sterilizes the input port 14.

The sterilizing unit 38 comprises a cap 42 that has a leading end 44, a following end 46 and an outside wall 48 extending therebetween. The leading end 44 has a first well 50 extending toward the following end 46. The following end 46 has a second well 52 extending toward the leading end 44. The outside wall 48 has an outside surface 54 and the outside surface 54 is threaded adjacent to the leading end 44.

The first well 50 insertably receives the nipple 30 on the syringe 22. The second well 52 has a bounding surface 55 and the bounding surface 55 is threaded. The bounding surface 55 threadably engages the outer surface 16 of the input port 14. Thus, the cap 42 is removably coupled to the input port 14.

A sponge 56 is positioned within the second well 52 and the sponge 56 is infused with the antiseptic 40. The antiseptic 40 may be rubbing alcohol or the like. The sponge 56 is comprised of a fluid absorbent material such as cotton or the like. The sponge 56 engages the outer surface 16 of the input port 14 when the cap 42 is coupled to the input port 14. Thus, the antiseptic 40 is deposited on the outer surface 16 of the input port 14 to sterilize the outer surface 16 of the input port 14.

A sheet 58 is provided. The sheet 58 is removably adhered to the following end 46 of the cap 42. The sheet 58 retains the sponge 56 in the second well 52. The sheet 58 is comprised of a fluid impermeable material.

In use, the syringe 22 is removed from the sterile bag. The cap 42 is positioned on the syringe 22. The sheet 58 is peeled away from the following end 46 of the cap 42. The cap 42 is manipulated to threadably engage the input port 14. Thus, the sponge 56 deposits the antiseptic 40 on the outer surface 16 of the input port 14.

The cap 42 is removed from the input port 14. The cap 42 is discarded and the second end 26 of the syringe 22 is coupled to the input port 14. The plunger 32 is manipulated to flush the intravenous line. The syringe 22 is discarded when the intravenous line is flushed. Alternatively, the cap 42 is left on the input port 14 to maintain the sterility of the input port 14.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous flushing system comprising:
an intravenous tube having an input port;
a cleaning unit being configured to contain a saline solution, said cleaning unit being selectively coupled to said input port wherein said cleaning unit is configured to urge the saline solution through said intravenous tube thereby facilitating said intravenous tube to be cleaned; and
a sterilizing unit being removably coupled to said cleaning unit, said sterilizing unit being infused with an antiseptic, said sterilizing unit engaging said input port wherein said antiseptic is configured to sterilize said input port.

2. The system according to claim 1, wherein said cleaning unit comprises a syringe having a first end, a second end and an outer wall extending therebetween, each of said first end and said second end being open, said syringe being configured to contain the saline solution, said second end selectively engaging said input port such that said syringe is in fluid communication with said intravenous tube.

3. The system according to claim 2, further comprising a plunger having a primary end and a secondary end, said plunger being slidably positioned in said syringe wherein said plunger is configured to be manipulated, said secondary end frictionally engaging said outer wall of said syringe such that said secondary end forms a fluid impermeable seal with said outer wall, said secondary end being urged toward said second end of said syringe wherein said plunger is configured to urge the saline solution into said intravenous line.

4. The system according to claim 1, wherein said sterilizing unit comprises a cap having a leading end, a following end and an outside wall extending therebetween, said outside wall having an outside surface, said outside surface being threaded adjacent to said leading end.

5. The system according to claim 4, wherein said leading end has a first well extending toward said following end.

6. The system according to claim 4, wherein said following end has a second well extending toward said leading end.

7. The system according to claim 5, wherein:
said cleaning unit includes a syringe having a second end;
said input port has an outer surface, said outer surface being threaded; and
said first well insertably receives said second end of said syringe, said second well having a bounding surface, said bounding surface being threaded, said bounding surface threadably engaging said outer surface of said input port.

8. The system according to claim 6, further comprising:
said input port having an outer surface, said outer surface being threaded; and
a sponge being positioned within said second well, said sponge being infused with the antiseptic, said sponge engaging said outer surface of said input port when said cap is coupled to said input port such that said antiseptic is deposited on said outer surface of said input port wherein said antiseptic is configured to sterilize said outer surface of said input port.

9. The system according to claim 8, further comprising a sheet being removably adhered to said following end of said cap, said sheet retaining said sponge in said second well.

10. An intravenous flushing system comprising:
an intravenous tube having an input port, said input port having an outer surface, said outer surface being threaded;
a cleaning unit being configured to contain a saline solution, said cleaning unit being selectively coupled to said input port wherein said cleaning unit is configured to urge the saline solution through said intravenous tube thereby facilitating said intravenous tube to be cleaned, said cleaning unit comprising:
a syringe having a first end, a second end and an outer wall extending therebetween, each of said first end and said second end being open, said syringe being configured to contain the saline solution, said second end selectively engaging said input port such that said syringe is in fluid communication with said intravenous tube, and
a plunger having a primary end and a secondary end, said plunger being slidably positioned in said syringe wherein said plunger is configured to be manipulated, said secondary end frictionally engaging said outer wall of said syringe such that said secondary end forms a fluid impermeable seal with said outer wall, said secondary end being urged toward said second end of said syringe wherein said plunger is configured to urge the saline solution into said intravenous line; and
a sterilizing unit being removably coupled to said cleaning unit, said sterilizing unit being infused with an antiseptic, said sterilizing unit engaging said input port wherein said antiseptic is configured to sterilize said input port, said sterilizing unit comprising:
a cap having a leading end, a following end and an outside wall extending therebetween, said leading end having a first well extending toward said following end, said following end having a second well extending toward said leading end, said outside wall having an outside surface, said outside surface being threaded adjacent to said leading end, said first well insertably receiving said second end of said syringe, said second well having a bounding surface, said bounding surface being threaded, said bounding surface threadably engaging said outer surface of said input port,
a sponge being positioned within said second well, said sponge being infused with the antiseptic, said sponge engaging said outer surface of said input port when said cap is coupled to said input port such that said antiseptic is deposited on said outer surface of said input port wherein said antiseptic is configured to sterilize said outer surface of said input port, and
a sheet being removably adhered to said following end of said cap, said sheet retaining said sponge in said second well.
